(19) United States
(12) Patent Application Publication
(10) Pub. No.: US 2020/0129109 A1
(43) Pub. Date: Apr. 30, 2020

(54) Mobility Assessment Tracking Tool (MATT)

(71) Applicant: Frank E. Bunn, Thornhill (CA)

(72) Inventors: Frank E. Bunn, Thornhill (CA); Jingbo Zhao, Toronto (CA); Constantine Papakonstantinou, Milton (CA)

(73) Assignees: Jingbo Zhao, Toronto (CA); Constantine Papakonstantinou, Milton (CA)

(21) Appl. No.: 16/174,727

(22) Filed: Oct. 30, 2018

(51) Int. Cl. A61B 5/00; A61B 5/11; A61B 5/103

(52) U.S. Cl. CPC A61B 5/4064; A61B 5/112; A61B 5/4023; A61B 5/7275; A61B 5/1038; A61B 5/7264; A61B 5/0077; A61B 5/0075; A61B 5/7425

(57) ABSTRACT

Mobility of a subject can reveal their physical, emotional and mental health, condition. The mobility assessment tracking tool (MATT) system assesses mobility derived by purely objective, reliable and reproducible processes and actions created by specialized computer codes and instructions executed by a logic engine to administer complex bio-mechanical measurements. The MATT system measures a subject's static and dynamic balance and gate mobility locomotion during the subject performing movements of the Tinetti gait and balance test and performing movements of the sports concussion assessment test, SCAT-5. By analyses of data streams from 3-D video with superimposed skeleton notes and from a pressure sensor force board used during these movements, the MATT assess a subject's mobility in accordance to established kinesiology definitions and protocol standards. Repetition of assessments over time allows for the tracking of the subjects' deterioration, maintenance or improvement health condition.

### REFERENCES

Tinetti, M. E., Williams, T. F. and Mayewski, R., “Fall risk index for elderly patients based on number of chronic disabilities,” American Journal of Medicine, vol. 80, no. 3, pp. 429-434, 1986.
U.S. Pat. No. 7,988,647 Aug. 2, 2011 Frank E. Bunn Class 600/595
U.S. Pat. No. 7,999,857 Aug. 16, 2011 Frank E. Bunn Class348/211.1
USPTO Patent App. 20060190419 Aug. 24, 2006 Frank E. Bunn Class 706/2
USPTO Patent App. 20100049095 Feb. 25, 2010 Frank E. Bunn Class 600/595
USPTO Patent App. 20140024971 Jan. 23, 2014 Frank E. Bunn Class 600/595
Bunn et al., “Gait Assessment Using the Kinect RGB-D Sensor”,
IEEE Engineering in Medicine and Biology, Milano, Italy, Aug. 25-29, 2015.
Wikipedia, Wii Balance Board with video game Wii Fit by Nintendo, Jul. 11, 2007
BJMS Online First—097506SCAT5, Apr. 26, 2017
York University, North York, Ont. Clinical Trials, Tinetti gate/balance test of athletes
Dr. Lauren Segrio, Dr. Diana Gorbet, Kinesiology, Jan. 2, 2016-Oct. 24, 2018

### BACKGROUND

The Faculty of Heath Sciences at Western University, London, Ontario, https://www.uso.ca, defines a person's ability to move or be moved as their mobility which forms a major outcome of human health and decreased mobility often occurs within the contexts of injury such as brain concussion or chronic diseases.

Bunn et al. U.S. Pat. No. 7,999,857 filed Jul. 25, 2003 revealed a computerized system including an intelligent camera and plurality of sensors which system analyzes video data viewing a scene of the movement of a subject to determine the subject's movement mobility and emotional stress to recognize the impairment level of the subject.

Bunn et al. USPTO application 20060190419 filed Feb. 22, 2005 revealed an intelligent video surveillance fuzzy logic neural network computerized camera system which system analyzes video data viewing the movement of a subject to determine the subjects facial and physical condition.

Bunn et al. U.S. Pat. No. 7,988,647 filed Mar. 16, 2009 revealed a computerized system including a video camera which system analyzes the video data of the movement of a subject to determine abnormalities in the subject's movement mobility from which comparison to known norms of mobility movement the system determines the subject's condition in relationship those norms. The application notes that results from comparison of the subject's condition to known abnormalities for diseases and illnesses those results may be used to generate treatment regimes.

Bunn et al. USPTO application 20140024971 filed Jul. 17, 2013 revealed a computerized system including a video camera which system using an active logic engine analyzes the video data of the movement of a subject to assess the mobility of the subject to determine mobility abnormalities, impairments, and there deterioration in the subject's mobility condition. The application notes that results from comparison of that subject's condition to known abnormalities and deterioration for conditions of brain concussion and diseases may be used to generate treatment regimes that lead to the restoration of the subject's health and thereby curing the condition.

The present invention relates to the objective computer video analysis computer code implemented by a computer coded logic engine, implementing a uniquely objective analysis computer code of the Mobility Assessment Tracking Tool (MATT) systems and objective computerized methods of determining and assessing the mobility of a subject performing movements and actions specified by Tinetti and SCAT-5 test protocols, through the administration of said code for computer vision objective analysis applied to 3-D video derived multi joint skeletal representation of the subjects' moving body parts and of the subject's foot pressure when performing on a force balance platform with pressure sensors. MATT fuzzy logic computer machine learning performs administration of complex bio-mechanical objective analysis assessments of the subject's static and dynamic balance and locomotion of said parts. The subject's kinematics of movement are measured, assessed and monitored by said objective analysis that derives mobility values from which the said code determines the level of the subject's assessed mobility functioning compared to normative values for said movement. Measurements and assessments of the mobility of the subject follow the kinesiology definitions established by the Tinetti test of gait and balance for risk of falling and the SCAT-5 concussion test. The Tinetti and SCAT-5 tests are originally subjective tests while here in revelled, the MATT is an objective computerize Tinetti test and SCAT-5 test. Boundary parameters for said measured kinematics may be adjusted as required, based on new and established best practices, for select populations including gender, age, athleticism, and disease or injury subgroups.

The present invention also relates to the objective computer video analysis computer code implemented by a computer coded logic engine, implementing a uniquely objective analysis computer code of the Mobility Assessment Tracking Tool (MATT) systems and objective computerized methods of determining and assessing the mobility of a subject through the administration of said code for computer vision objective analysis applied to 3-D video derived multi joint skeletal representation of the subjects' moving body parts and said code for a subject's foot pressure objective analysis applied to the pressure sensor data derived from a pressure force platform said sensors detecting the force of the subject's feet when performing movements on the platform. MATT fuzzy logic computer machine learning performs administration of complex bio-mechanical objective analysis assessments of the subject's static and dynamic balance and locomotion of said body parts and said foot pressure. The subject's kinematics of movement are measured, assessed and monitored by said objective analysis that derives mobility values from which the said code determines the level of the subject's assessed mobility functioning compared to normative values for said movement. Measurements and assessments of the mobility of the subject follow the kinesiology definitions established by the Tinetti test of gait and balance for risk of falling and by the SCAT-5 concussion test. The Tinetti and SCAT-5 tests are originally subjective tests while here in revelled, the MATT modifies these test to be objective, reproducible, computerize tests. Boundary parameters for said measured mobility assessment may be adjusted as required, based on new and established best practices, for select populations including gender, age, athleticism, and disease or injury subgroups.

The MATT is designed to save time for both administrators and health care professionals. Mobility assessments results provide gross overall values, scores and detailed results of a subject's performance of movements. Provided in a readily accessible test format, results can quickly and easily be recorded within computer database frameworks based on kinesiology practice and protocol, and output in printable standardized kinesiology report formats of the numerical and textural mobility assessment results and recommendations.

### BRIEF SUMMARY

In general terms, the present invention provides a system, the Mobility Assessment Tracking Tool (MATT), for objective computerized analysis assessing the mobility of a subject, said system comprising: two or more motion sensors to observe movement of a subject performing 8 simple movements and to generate and record a 3-D video digital data stream representative of such movements. An active logic engine administering computer vision technologies including machine vision and machine learning functions apply MATT objective computer code implemented by a said logic engine analysis to determine and record from the video a multi-nodal skeleton representation of the physical joints of a subjects' body parts movement for each video frame to frame of the moving subject which representation is isolated from the stationary background. A set of fuzzy logic computerized code instructs said active logic engine to apply machine vision objective code such that for each measurement, each with one or more adjustable parameters, can be administered for interpreting the kinesiology defined kinematics of each body part movement within which the movement can be determined by the said engine administering machine learning logic of the objective code, the measure of level of function of the movement to be lying within or lying outside of normative range of values of specific features' values of the movements. The administration of further objective code by the engine to these features' values by which the said further objective code can determine the mobility assessment of the subject. Further additional objective code provides automated output of assessment results as a readily accessible text format for standardized reports in numerical and text interpretations of the assessment.

Through computerized automation the MATT provides consistent, reliable and reproducible mobility assessment results across testers administering the MATT tests of a subject's mobility. Every subject receives identical computerized verbal and video instructions each time they perform the assessment test thereby eliminating tester and intra-tester reliability as a source of error. Verbal instructions can be provided in a variety of languages to suit the subject being assessed.

The MATT is designed to save time for both administrators and health care professional. Assessment results provide both gross overall mobility scores and detailed results of the subject's performance of test movements. Here-to-fore such assessments have been made in a subjective assessment by kinesiology professionals. The MATT provides these assessments in a computerized objective, repeatable and reliable system utilizing objective analysis determining said assessments. Provided in a readily accessible text and numerical formats, the results can be recorded within internal system frameworks, based on practice setting. These formats provide to mobility practitioners, automation efficiency reducing their time to make and report a subject's mobility assessment while increasing the consistency of those assessments.

The MATT tool provides to mobility practitioners, a computer-automated, objective, reproducible and reliable assessment in keeping with kinesiology standards of measurement of the mobility of a subject and potential related medical conditions and remedial procedures, providing the critical information the practitioner needs for diagnosis of the subject's condition, injury, illness, or affliction and the treatments that may be needed.

In a further aspect, the invention provides a method of assessing mobility of a subject comprising the steps of recording motion of said subject, administering fuzzy logic machine vision and machine learning computer code applied with said active logic engine, on said motions to determine kinematic assessments of mobility and for determination of abnormalities of such movement, determining relationships of said abnormalities to known normative values, and determining whether said abnormalities are within a known norm or range of known norms.

In a further aspect, the invention provides methods and systems of administering an allocator on said active logic engine to determine if said abnormalities are within a known normative value or range of known normative values whereby to determine the possible existence of bio mechanical or neurological conditions or injuries of the subject and to determine at what stage are the said conditions or injuries. The invention further can infer from these determined conditions or injuries what are their relationships to known kinesiology rehabilitation procedures and treatments for such conditions or injuries, and can determine the potentially appropriate rehabilitation procedures recommended by the said methods and systems to relieve, repair or restore the subject's health and potentially cure the said conditions or injuries.

From the above, it will be clear the determination of mobility impairment will include the deterioration of the walking gait of a subject. It has been shown by extensive studies that the deterioration in mobility, including gait, of a subject has been directly correlated to neurological deterioration of the subject. Dr. Dean M. Wingerchuk at the Mayo Clinic in Rochester Minn. has reported “Gait analysis adds objective, reliable outcome measures sensitive to detecting neurological deterioration.” Neurological deterioration can also be caused by brain concussion for which the SCAT-5 test was specifically designed to detect. Dr. Wingerchuk states that “Gradual deterioration in ambulatory function is one of the major manifestations of progressive forms of Multiple Sclerosis”. At the Alzheimer's Association International Conference 2012 in Vancouver, Canada, three independent research studies each surveying more than 1,000 people, all confirmed mobility deterioration in gait of subjects directly reflected their neurological deterioration due to their Alzheimer's dementia. The studies were conducted by Dr. Stephanie A. Bridgenbaugh of the Basel Mobility Center in Basel, Switzerland; Dr. Mohammad Ikram at Erasmus MC Rotterdam, the Netherlands; and Dr. Rodolfo Savica of the Mayo Clinic Study of Aging, Rochester Minn.

From the above, it will be clear the assessment methods and system means described could be applied to the determination of mobility impairment including the deterioration of the walking gait of a subject to determine the potential existence of brain related illnesses including but not limited to Multiple Sclerosis and Alzheimer's dementia and brain concussion.

In this example, the expert system administers the said objective computer code by the active logic engine to the data available to identify that a mobility impairment condition exists in one or more movements in the current assessment and accesses a data base to determine relationships of this mobility impairment condition to a previous assessment for this subject, stored in the database component of this system, to determine if this mobility impairment condition was detected in a previous assessment. If the mobility impairment condition did so exist, the computer system, administering time derivative determinations, calculates the rate of change in the mobility impairment condition between successive assessments for this subject. The computer facility, using a predetermined baseline matrix of outcomes, then determines if a critical mobility impairment condition exists and, comparing to previous assessments, determines if a deterioration in the mobility impairment condition has occurred, and if so occurring computes the rate of change of this deterioration. This said objective computer code of the active logic engine of the MATT computer system can be applied to the assessment of brain concussions and conditions of the subject as is discussed herein.

### DESCRIPTION OF DRAWINGS

Embodiments of the invention will now be described by way of example only with reference to the accompanying drawings in which:

FIG. 1 is a schematic of a 3-D representation of a dual camera observation of the sit-stand-sit movement mobility assessment of a subject.

FIG. 2 is a schematic 3-D representation of a dual camera observation of the turn-in-a-circle, turn-on-the-spot, and stumbling, movements for mobility assessment of a subject.

FIG. 3 is a schematic representation of a wobble movement functional assessment process for a wobble forwards, backwards, or possibly side to side.

FIG. 4 is a schematic representation of wandering deviation from normal movement mobility assessment of a subject illustrating wander from kinesiology walking standards for a straight normal path (upper plot) and for wander from normal foot-to-foot separation (lower plot).

FIG. 5 is a 3-D plot of left and right foot measurements that are made by the Mobility Assessment Tracking Tool System with definitions of the features measured in the gait assessments.

FIG. 6 is a spreadsheet example of all the mobility assessment features measured for fourteen selected athlete subjects during the Walk and during the Turn-360 Degrees movements.

FIG. 7 is a spreadsheet example of the Thresholds established for each of the mobility assessment features measured for the Walk and the Turn-360 Degrees movements.

FIG. 8 is a schematic representation example of three stances for a subject standing on a pressure sensor board such as a WiiBoard.

FIG. 9 is a schematic representation of analysis processing for MATT use of the WiiBoard data.

FIG. 10 is a schematic representation of the deduction flow diagram of the center of pressure, COP, assessment of step-stumble or heel-toe lift by MATT analyses of WiiBoard data.

FIG. 11 is a schematic representation of the deduction flow diagram of the center of pressure, COP, assessment of step-stumble or heel-toe lift for MATT analyses of WiiBoard data viewed as a rotational transform.

FIG. 12 is a schematic representation of the deduction flow diagram of the center of pressure, COP, assessment of step-stumble or arch-lift for MATT analyses of WiiBoard data viewed as a rotational transform.

FIG. 13 are three 2-D plots over 25 seconds of time as calculated from Wii board data for: top plot, a double leg stance test illustrating the right and left sagittal sway of a subject; middle plot the forward and reverse coronal sway of a subject; and bottom plot the angle of the center of pressure, COP, of the subject.

### DETAILED DESCRIPTION

Prior to describing the system and its function in assessing mobility of a subject from observing the subject performing 8 specific movements: sit still in a chair; arise from a chair; stand still; stand still with eyes closed; walk in a straight line path; turn 360 degrees walking in a circle; and turn 360 degrees walking on-the-spot. A number of the typical assessment environments will be described to provide context to the operation of the system.

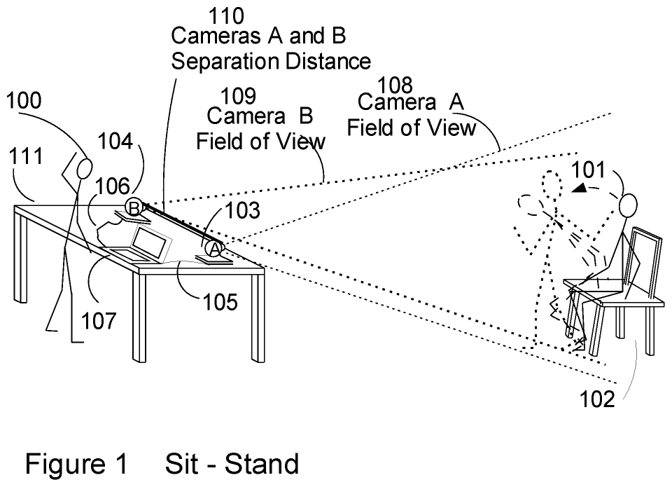

Referring to FIG. 1, an expert system apparatus is used within a typical professional office environment for observing and video-recording specific movements of a subject, (101). The system includes a computer (107) that implements an active logic neural networks decision engine, to administer the said objective computer code by the active logic engine to video data obtained from motion sensors (103 and 104). The motion sensors may be a camera or cameras operating in one or more of the visible, or infrared, or ultraviolet spectrum, an acoustic image capturing device or location sensors such as GPS positioning devices or RF motion/location devices from which to generate and record information of the movement of the subject. For convenience they will be collectively referred to as cameras. The expert system embedded in the computer (107) operates on and administers said objective computer code by the active logic machine vision logic engine to the video data stream from the cameras (103 &104) to derive and record a multi-joint skeleton nodal data stream with each node representation of one of the joints of the subject' body in each frame of the video. Administration to the skeleton representation, of additional active said objective computer code by the logic engine machine learning tests enable the expert system to determine and record 13 specific features' values measured, to be described later, of the subjects' movement and determines whether the movements observed are an abnormal condition, that is, one that departs from expected or desired kinesiology standards of motion and commonly referred to as normative or normal motion. The system utilises that condition information to assess a particular condition, such as presence of a bio mechanical injury or neurological injury causing the limited level of compliance to the kinesiology standards for that movement.

In FIG. 1, a subject (101, solid lines) sitting in a chair (102) is being observed by a cameras (103 & 104), connected via wired or wireless interfaces (105 & 106) to the computer (107) being operated by a test facilitator (100). The test procedure conducted provide computerized voice instructions to the subject, requiring the subject to sit upright, straight and steady in the chair and the administration of the additional said objective computer code by the active logic engine to the derived the skeleton nodal data stream from which 13 specific features' values of the movements are determined as representation of the movement of the subjects' body and further said objective computer code administration by the engine to the set of feature values will detect any abnormal position or movement of each body joint of the subject represented by a node. The subject is then requested by the computerized voice to arise and the administration of the additional said objective computer code by the active logic engine to the skeleton nodal data stream and derived specific features” values representative of the rising movement of the subjects' body will detect any abnormal motion of that movement of subject arising from the chair from the measuring the 13 specific features' values in a process that will be detailed later herein. The cameras (103 & 104) detect the motion of the subject (101) and the expert system transfers and records the data representing the motion to the computer (107) for further processing.

As an example, say the subject takes two attempts to rise from the chair (101, dotted lines). The cameras (103 &104) capture the movement of the subject (101) in a time dependant manner and the data are transferred to the computer (107). As will be described more fully below, the expert system administers the new and uniquely developed mobility assessment movement said objective computer code instruction to the said logic engine being revealed herein, which will be referred to as the mobility code to determine normality or abnormality of the movement according to kinesiology standards of movement as derived from the 13 specific features' and applies this information and additional input to provide the criteria required to apply standardized kinesiology test criteria and test parameters. In the example provided, the two attempts to rise are determined as an abnormal mobility condition and these determinations indicate that the subject has a significant limited level of compliance to the kinesiology standards for that movement and defining the subject's impairment condition for that movement.

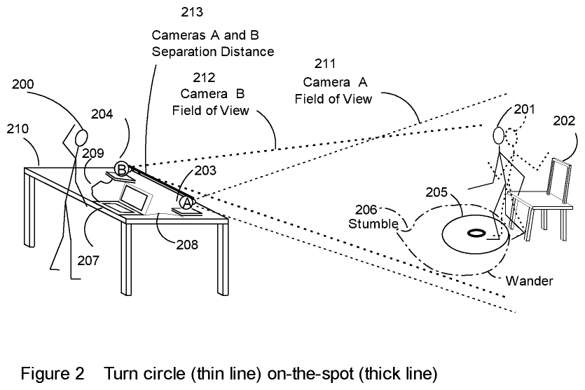

FIG. 2 shows a typical functional test assessment process and decision computations for a subject (201) having risen from a chair (202), to stand still, then turn around 360 degrees. The test facilitator (200) and the computerized voice instructions asks the subject, to stand still for assessing steadiness without wobbling or swaying, The computer (207) and the cameras (203 & 204) capture and record the video data of the movement indicated at (201), where solid lines stick-person subject and dotted lines stick-person subject indicate change of position over time to indicate that the subject is wobbling. In this example, the expert system, administering the mobility code by the logic engine in real time or to the recorded data, may determine the wobble or swaying as being a limited level of compliance to the kinesiology standards of mobility for that movement. These determinations are provided to the selected established kinesiology standards for those movement test procedures and mobility scoring, and, depending on the cumulative results, the expert system may decide the subject has a significant level of difficulty performing that movement (201). The expert system also determines the level of mobility of the subject's actions while standing (201), wherein wobbling, swaying or stumbling is detected, recorded and scored.

Continuing with this FIG. 2 example, the computerized voice instructions then asks the subject to turn 360 degrees in a circle along the path (205), for which the solid line indicates the expected circular track for normal turning. The expert system observes the actual movement (206) indicated by the dotted line and administers the mobility code by the active logic engine to the sensor data to determine the wandering and stumbling as being a limited level of compliance to the kinesiology standards for that movement. This is input into established test procedures and mobility scoring to determine if the subject has significant mobility limited level of compliance to the kinesiology standards for that movement for that movement.

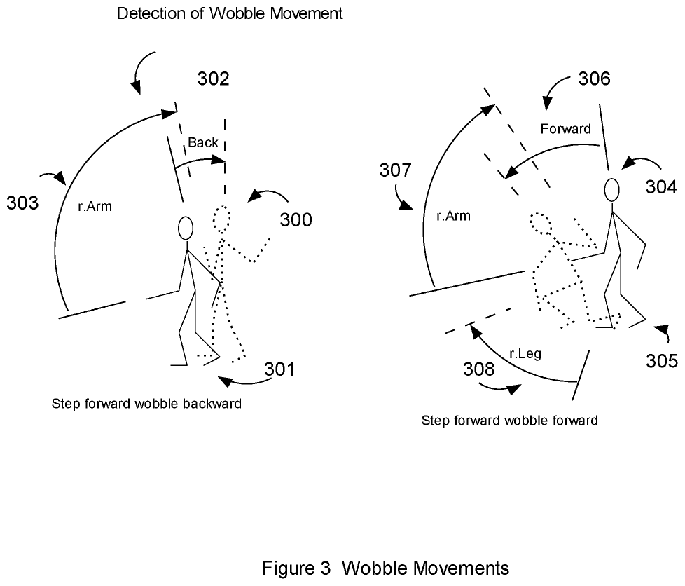

Eight kinesiology accepted movements have been selected that are used to observe and assess mobility, the occurrence of mobility impairment and conditions of a subject and the subject's potential of having related injury, illness, pain or disease for that subject being assessed. FIG. 3 illustrates examples of two movements of a subject which would normally be determined by the mobility code assessment to deviate from expected normal or standard movement. These are the step forward (301) from start position (300) indicating wobble back (302) and arm movement 352 (303); and the step forward (304) from start position (305) indicating wobble forward (306) and arm movement (307) and right leg swing (308).

Normal for a specific subject means movement that has been previously observed and recorded in databases for this subject and is accepted as a base level of compliance to the kinesiology standards for that movement. Standards for that movement can be defined as movements that have been observed and recorded in databases of typical movements for subjects of similar age, sex, health, and mobility and is accepted as a base level of compliance to the kinesiology standards for that movement for any similar subject.

The mobility codes revealed in this invention are administered by the active logic engine expert system, to the input video data streams from a multiplicity cameras to derive the skeleton nodal data streams and to derived specific features' values data streams, said additional mobility codes functioning as an administrator, to conduct detection determinations, and specific features' extraction from the nodal data stream administrations, from which to assess the likelihood of limited level of compliance to the kinesiology standards for that movement for a subject. This is accomplished by administering the mobility codes by the active logic engine to video data, to develop for each frame of the video data stream a computerized frame by frame skeleton nodal data stream representation of the subjects' body including multiple control joints such as: head, neck, shoulders, elbows, wrists, hands, torso, hips, knees, ankles and feet. Further mobility codes are administered to each skeleton nodal representation for each frame to determine a measurement of specific features' values of the movements of each joint relative to their location in the previous frame. Additional mobility codes are administered to each measurement to determine metric amount of that joint's movement where by the mobility codes can determine the bio mechanical movement of the subject's body at each joint. For example specific features' movements values such as for feet movements: step length, height of moving foot off the floor, separation between feet, step frequency can be determined. Another example for arm specific features” movements relative to: shoulder, elbow, wrist and torso joints, the angle of the upper arm and lower arm relative to the position of the torso can be determined from the angles formed by the wrist-elbow-shoulder joints. Not all such movement examples will be discussed here but it will be clear to any one informed in bio mechanics that with sufficient control joints, most bio mechanical body movements can be determined.

These specific features' values as determined by administration of the mobility codes described above, also produce electronic or mathematical signatures of said movements such that administration of additional mobility codes can derive from these movements, an allocator value to determine whether the values of said signatures are within known norms of the movement of personal, and/or, normal range level of compliance to the kinesiology standards for that movement and deviations there from for features' movement of normal subjects which provide features' signatures of movement are stored in the system in related databases. Then, deriving similar signatures of subjects to be assessed as to mobility performance of the movements, the active logic engine mobility codes determine the deviation of these signatures from the normal signatures to make the decisions as to infer limited level of compliance to the kinesiology standards for that movement. If limited level is interpreted, the mobility codes then determine whether the movement indicates a bio mechanical or neurological injury, pain, or illness and if so indicated, it informs the appropriate health care personnel or systems. Similarly, determinations of the deviation of subject's movements could result from medical emergencies such as heart attack, or seizure that such emergencies also require healthcare personnel assessment in responding to the subject in question for which appropriate medical actions can be taken.

The administration of the mobility codes of the system using the active logic engine, can implement unique determinations and subsequent reporting assessment results for mobility level of compliance to the kinesiology standards for that movement. These reports can be in readily accessible text format that can be cut and pasted into internal and external standardized reports based on kinesiology practice. Later, such observations of the subject will determine the changes in the subject's movement as it correlates to their earlier determinations and in real time determine any deviations that could relate to mobility reduced level of compliance to the kinesiology standards for that movement and possible existence of injury, pain or medical health condition as determined by the active logic engine mobility codes. However, if the mobility codes administration system through access to related databases has access to medical and health information and database of related mobility impairment signatures of the subject, the active logic engine processor may be able to determine if the subject being observed is in fact having a health problem such as heart attack, stroke, diabetic coma, epileptic seizure or brain related diseases such as Multiple Sclerosis, Parkinson's, Dementia, Cerebral Palsy, or brain concussion, and any of which could be needing immediate medical assistance and if so determined, can inform the proper health care providers.

In the case for that a subject is determined to have a reduced level of compliance to the kinesiology standards for a movement, for example as a stagger back shown in FIG. 3, the subject in attempting to step forward (301 solid line stick figure), actually staggers backward (300 dashed line stick figure) in which the major motions of the subject's back (302) and right arm (303) could be determined by the mobility impairment assessment mobility codes administered to the specific features' values data stream, to have deviated from expected for either the normal or standard movement. Similarly a stagger from side to side could indicate impairment. In the stagger forward example, the subject in attempting to step forward (304 solid line stick figure), actually staggers forward (305 dashed line stick figure) in which the major motions of the subject's back (306) and right arm (307) and left leg (308) could be determined similarly by the mobility impairment mobility codes to deviate from expected for either the normal or standard movement. Details will be discussed later.

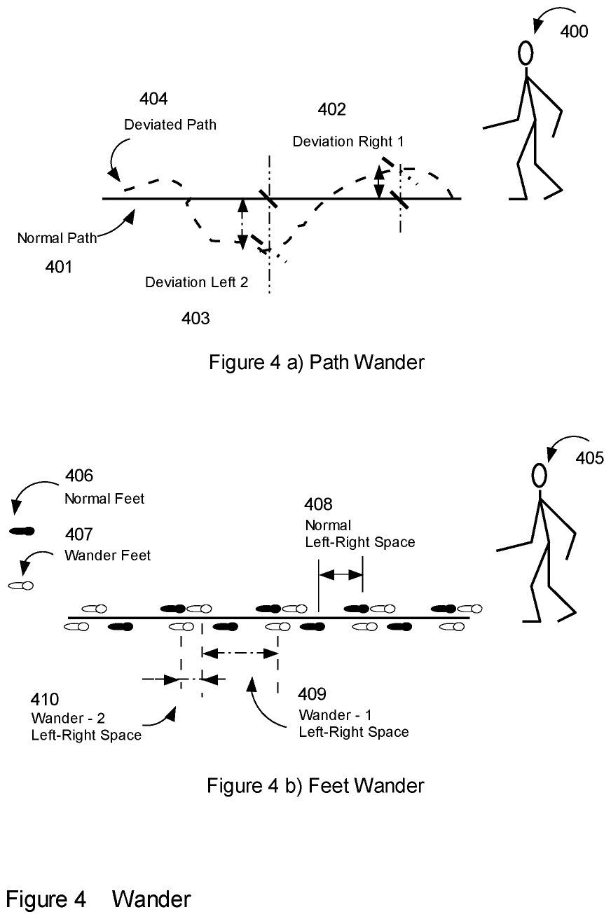

FIG. 4 a) illustrates movements of (400) a subject's feet, normal (406) or wander (407), in which the subject's walking path wanders from a normal or standard path (401) for the subject's feet indicated by a Deviation Right 1 (402) and a Deviation Left 2 (403) which would be determined by the administration of walking mobility codes to the skeleton nodal data stream of control joint data to deviate from expected for either the normal or standard movement. Further, FIG. 4 b) illustrates specific features' movements derived from the skeleton nodal data stream of (405) a subject's feet which wander from the expected normal (408) or standard foot spacing where the subject's left to right Wander-1 (409) spacing is larger than expected and right to left Wander-2 (410) spacing is shorter than expected. The unexpected movements could be determined by the mobility level of compliance to the kinesiology standard movement's mobility codes to deviate from expected for either the normal or standard bio mechanical movement. Further details will be discuss later.

Further, a significant foot placement specific features' test while walking is to request the subject to walk toe-to-heal such that the subject places each foot at each step so that the heal of the front foot touches the toe of the back foot. This is a more difficult and perhaps stressful walking task for the subject and the mobility assessment of the subject's movement can determine more subtle effects of and existence of bio mechanical or neurological problem. Further, an even more difficult walking task is to request the subject to walk either regular walk or toe-to-heal walk but with the moving foot to cross over the stationary foot such that the subject's feet when both are stationary are crossed at every step in the walk. Mobility assessment of the subject, under the stress in this task, can determine even more subtle effects of and existence of bio mechanical or neurological problems. It will be obvious to anyone verse in bio mechanics, that many more movements will be applicable for administration of the mobility codes revealed herein for mobility assessment, however for brevity are not detailed here.

The above examples relate to an assessment performed in a controlled environment by a medical practitioner, tester or operator. The MATT system incorporates computerized voice instructions for each movement the subject is requested to perform thereby providing consistent reproducible test procedures. The expert system may also be used in a normal non-clinical environment as a continuous, non-invasive mobility assessment tool, such as a mobile computer and cameras system implemented near an athletic playing field to provide quick on-sight assessment of athletes before, during or after play. Particularly if a player is suspected of having suffered a hit, shaking or injury to the body during play, a prompt mobility assessment at the time of such occurrence could be critical in assessment for potential bio mechanical or neurological problem and the expert system mobility codes could be administered to alter health providers and practitioners such that immediate action for medical attention can be taken as needed.

The Sport Concussion Assessment Tool, now in version #5, BJSM Online First Apr. 26, 2017-097506SCAT5, is an established, professional kinesiology subjective test for the assessment of the mobility of sports participants expected to have suffered such a hit to the body that may have produce the potential bio mechanical or neurological problem resulting from brain concussion. Herein we reveal a purely objective computerized implementation of SCAT5 using an instrumented force balance board. One such board is the WiiBoard balance board for the game console Wii Fit by Nintendo, Wii balance board referenced in Wikipedia. The MATT has integrated pressure data from the WiiBoard data into the MATT system for collection of pressure data from the pressure sensors located on the four corners of the board. Additional mobility codes of the system using the active logic engine are implemented for accessing the Wii data and integrating these data into the MATT system.

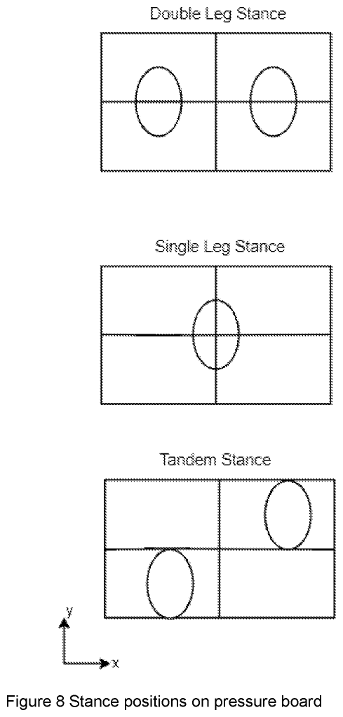

Further additional mobility codes of the expert system analyze these data to determine the balance and foot pressure on the board by the subject preforming the movements required for the SCAT-5 test. The MATT system expert system analysis mobility codes also records the video of the subject's movements on the Wii board. Additional mobility codes of the system using the active logic engine are implemented to integrate and analyze the Wii board balance and pressure data. The MATT expert system also records the video of the movements of the subject conducting the required SCAT-5 movements on the balance board including but not limited to: standing on both feet, standing on one foot, standing extending lifted leg, standing toe to heal. FIG. 8 is a schematic representation of examples of three stances positions of a subject standing on a pressure sensor board displayed on a quadrant representing the force sensors on the board.

The MATT system additional mobility codes using the active logic engine extracts and records the following four specific features from the analysis of the Wii board data. The sagittal sway which is defined as the sum of the weight ratio of adjacent left and adjacent right Wii board sensor plates which are separated by the sagittal plane. The coronal sway which is defined as the sum of the weight ratios of the anterior and posterior sensor plates which are separated by the coronal plane. The diagonal sway which is defined as the sum of the weight ration of the sensor plates on each diagonal of the Wii board. And the center of pressure, COP, which is defined as the center of the pressure distribution over all sensor plates on the Wii board.

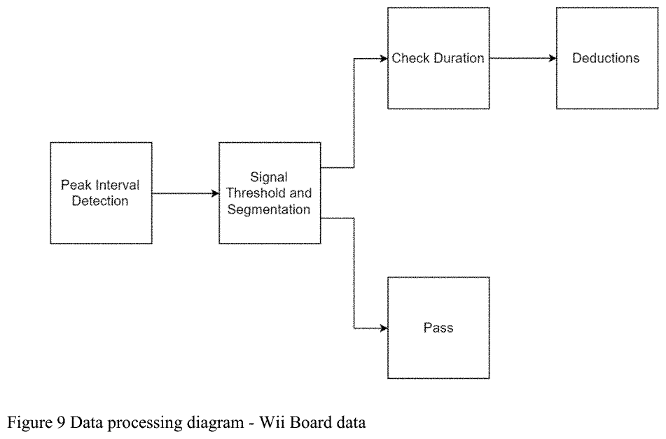

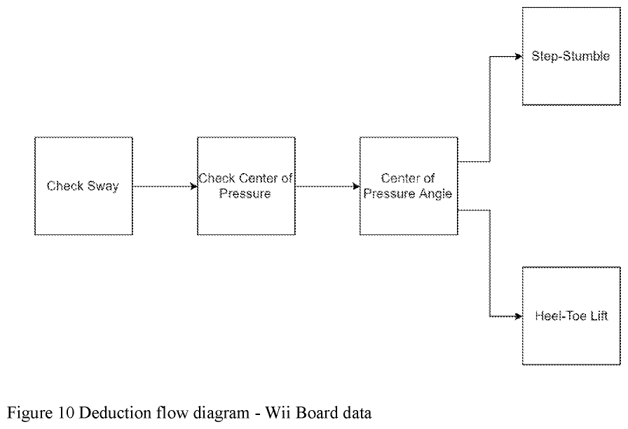

The measured values from the Wii board sensor data for these four specific features are analyzed by the additional mobility codes using the active logic engine processing as illustrated in FIG. 9. These analyses are correlated to the video stream data mobility assessments to determine the SCAT-5 balance test assessment score for the potential of brain concussion for the subject being assessed. The deduction and assessment of the duration of any loss of balance as illustrated in FIG. 10, is determined as the subject's stance, and the type of balance loss including sway, is determined as a step, or as a stumble, or as a heel-to-toe lift. Additionally, the MATT video data stream and skeleton data stream are analyzed to identify hip abduction and hand lift off the subject's hip area of the Illiac crest.

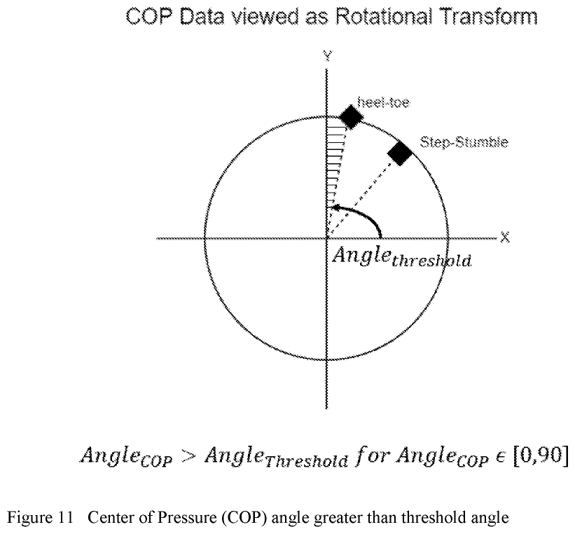

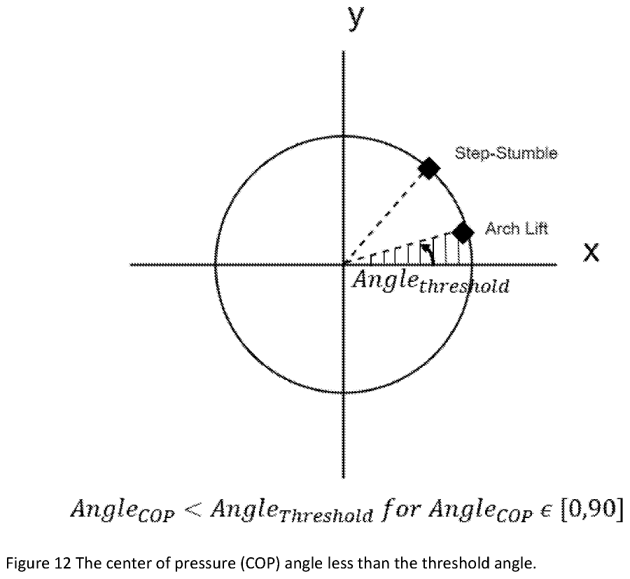

Stepping and stumbling are analyzed from the Wii board data by computation of the rotational transformation about the center of pressure. A threshold value establishes the boundary values by MATT based on established clinical data to determine the boundary between stepping-stumbling and heel-to-toe lift as illustrated in FIGS. 11 and 12.

The center of pressure angle can be calculated as:

$$\begin{matrix}{r}_{COP}^{2} = {x}_{COP}^{2} + {y}_{COP}^{2} & ( 1 ) \\ {Angle}_{COP} = {sin}^{- 1} ( \frac{y}{{r}_{COP}} ) OR {cos}^{- 1} ( \frac{x}{{r}_{COP}} ) & ( 2 )\end{matrix}$$

The criteria used for scoring in the balance testing related to the Wii board are:

Heel—Toe Lift: which we defined as a regime of angular positions from the patient stationary reference frame, which has the Wii board coordinate system as it's inertial reference (See FIG. 11);

Step Stumble—which we defined as a regime of angular positions between Heel-Toe (See FIG. N3), and Arch lift (See FIG. 12) from the patient stationary reference frame, which has the Wii board coordinate system as it's inertial reference (See FIGS. 11 and 12);

Duration: The period of time for which the peak of signal which exceeds a sway or center of pressure threshold.

The values in conjunction with the video stream data are used to satisfy the objectives of the SCAT-5 Balance testing. Deductions from the Wii board data are assigned based on duration of loss of balance (stance), and type of balance loss; step, stumble, or heel-toe lift (See FIGS. 9 and 10). MATT video stream and skeleton data are used to identify hip abduction, or if hand lift off Iliac crest.

For each of the stance positions (See FIG. 8), the balance signals are monitored for peaks in balance loss, and differentiation between stepping-stumbling, and heel-toe lift can be determined from the Wii board data by the rotational transformation upon the center of pressure. A threshold is applied by MATT to determine the boundary between stepping-stumbling, heel-toe lift and arch lift (See FIGS. 11 and 12). MATT may also corroborate data from the video feed for determination of the nature of the score deduction via its nodal skeleton analysis. MATT further uses the clinical data to identify a threshold for distance of the wrist from Iliac crest, and a threshold of 30 degrees from nominal stance as defined in SCAT-5 for deductions associated with hand lift from iliac crest and hip abduction.

The three nominal test stances as defined by the SCAT-5 methodology (See FIG. 8) are defined as:

Double Leg stance uses the Sagittal, and coronal sway data to search for peaks, and the nature of score deduction is determined by observing the center of pressure angle during the interval for which there was a peak greater than the threshold values for balance loss;

Single Leg stance uses the Sagittal, and coronal sway data to search for peaks, and the nature of score deduction is determined by observing the center of pressure angle during the interval for which there was a peak greater than the threshold values for balance loss; and

Tandem stance uses the diagonal sway data to search for peaks, and the nature of score deduction is determined by observing the center of pressure angle during the interval for which there was a peak greater than the threshold values for balance loss. In this test, the patient reference coordinate frame is not parallel with the inertial reference frame.

MATT also incorporates the mobility assessment values from the video and skeleton data streams observations and mobility assessment of the subject's movements on the Wii board, to determine the nature of the SCAT-5 balance test score. MATT further establishes the boundary values based on the established clinical data to determine the boundary thresholds for the distance of the wrist from Iliac crest and for the angular raise of the wrist from established values for nominal stance as defined in the SCAT-5 test for deductions associated with hand lift from Iliac crest and hop abduction.

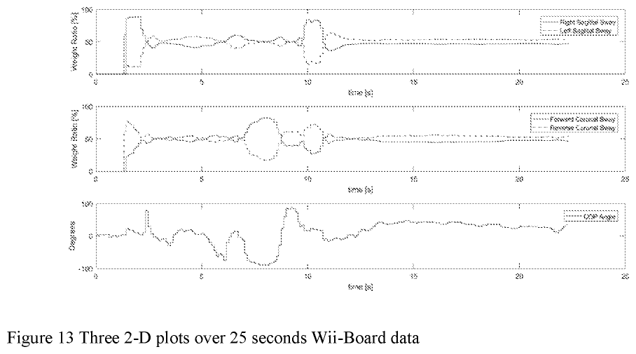

FIG. 13 illustrates the comparison of data calculated from Wii board over a 25 second time interval. The top plot show the right and left sagittal sway of a subject; this data indicates if the subject has lost his balance on the left or right side of the body sagittal plane. The middle plot shows the forward and reverse coronal sway of a subject; this data indicates if a subject has lost his balance to the front or back of the body coronal plane. The bottom plot shows the angle of the center of pressure, COP, of the subject, comparing balance loss with this angle reveals if the balance loss was due to a heel-toe lift, or a step-stumble.

The MATT expert system mobility codes determining the subjects stance and balance for these video data mobility assessments further determines the SCAT-5 test scoring. Vocal response and vision response SCAT-5 tests are administered by additional expert system mobility codes from which the expert system determines the subjects scoring of cognitive SCAT-5 data tests.

The MATT expert system mobility codes integrate the scoring results from the video data assessments and from the cognitive data assessments from which to objectively determine the assessment of the potential that the subject suffered a brain concussion. The balance and foot pressure analyses are integrated with the MATT mobility video analyses, assessments for the participant subject, from which analyses MATT expert system mobility codes derive the unique determinations and subsequent assessments results for the subject's mobility level in compliance to the kinesiology standards for the SCAT-5 test.

The implementation of the expert system can be considered as having two main linked components: a basic mobility assessment system and an advanced mobility assessment system. The basic system permits an operator to control part or all of the assessment process and to input assessments of the mobility of the subject being assessed. The advanced system contains the mobility codes and computer facility active logic engine neural networks decision computations with which the expert system determines the assessment outcomes and recommendations according to established parameters, the mobility assessment total score number, and the differential determination of current assessment to previous assessments, and generates reports of remedial actions, possible aids and healthcare procedures, to the subject, or to the subject's employers or to the caregivers of the subject.

Further, the expert system may be administered using a limited number of skeleton nodal control points such as head, shoulders, trunk, elbows, wrists, hands for monitoring larger arm movements. Alternatively the expert system could also use a larger number of control points including the above plus thumbs, fingers, knuckles for refined higher resolution of movements such as for observing shaking of hands that could be typical of diseases such as Parkinson's.

The advanced system can compute a larger number of skeleton nodal control points and related selected specific features' values assessed than does the basic assessment system, for each video frame. Using known video skeleton nodal control point to create additional points, the advanced system can then derive additional specific extracted features' with which to detect the finer more precise subject's movement of each control point from frame to frame based on the displacement of each control point on a given frame relative to the same control point on the previous frame by differentiating between those two to determine the control points that are moving and those that are stationary on a frame to frame basis. Extracting said features applies to a subject's movements made while standing on the balance board and to the subject's movements made while not standing on the balance board.

Extracting said features may be performed by the MATT objective computerized analysis mobility codes determining a specific extracted feature such as whether a given skeleton nodal control point, E for example of an elbow movement, in the image frame, x, moves or is displaced by or more than say 3 video pixel spaces in any direction for this control point in its location in the next image frame, y. If so then this control point, E, in frame x is identified by the MATT objective computerized analysis mobility codes as moved and assigned pixel component location. If pixel E in frame x, moved less than 3 pixel spaces at its new location in frame y, then this control point, E, is identified as not moved and assigned the pixel components it had in frame x. By the MATT objective computerized analysis mobility codes computing the movement of all control points from frame x to their locations in frame y and assigning all those that move 3 or more spaces, with the new pixel locations where they appear in frame y and all those control points in frame x that move less than 3 pixel spaces to retain their pixel locations from frame x, a skeleton motion-rendition of the subject's movements wherein all movement of the subject can be observed and movement assessed by the MATT objective computerized analysis mobility codes. The number of pixels, for example here being 3 or more, is set by adjustable additional mobility codes pixel parameter by which the administration of the pixel movement MATT mobility codes determines the number of pixels moved. Additionally, administration of the MATT pixel movement mobility codes to the 3-D data stream components of the cameras, the MATT objective computerized analysis mobility codes can determine the physical distance of the skeleton nodal control joint movement from frame to frame where the distance of the movement is set by an adjustable additional mobility codes distance parameter input to the administration of the mobility codes.

The finer movement and measurements resulting from the higher number of skeleton nodal control points can be considered as a higher resolution detection skeleton nodal data stream and derived specific features' values representation which in this case is the subject being mobility level of compliance to the kinesiology standards for that movement assessed, and stores that skeleton nodal data steam and derived features' values representational data in a database. It is preferred that the mobility impairment detection mobility codes revealed herein are advances on and entirely new derivations of those stagger computer codes which only consider detection of the movement of the envelope shape of the entire body of a subject revealed in U.S. Pat. Nos. 7,988,647, and 7,999,857, and networking computer codes of U.S. patent application 20060190419 and determination of medical conditions by measuring mobility patent application 20100049095, and assessment and cure of brain concussion and medical conditions by determining mobility patent application 20140024971, the contents of which are incorporated herein by reference.

However the mobility codes revealed in this patent application are completely new. The mobility codes of this application objectively observe, measure and assess the movement of the individual body parts by measuring and tracking the movement of the skeleton nodes and assessing the pressure sensor observations of the subject performing movements while standing on and while not standing on a balance board, indicated earlier, for determinations of the assessment of mobility and mobility impairment and potential of brain concussion of the subject.

By using such techniques, it is possible to evaluate if a particular movement is indicative of a mobility level of compliance to the kinesiology standards for that movement and if an impairment condition exists from determining the movements of a subject. Each of these evaluations may be made from the specific extracted features' values derived from the skeleton nodal data stream of the motion by determining the average deviation of a set of specific features' data representing the body, for example determining the average location of the centreline of the subject relative to the normal path for that movement.

The mobility assessment mobility codes are administered to the real-time or recorded video data stream and to the associated skeleton nodal data for an objective determination of the 13 specific features' values measure of the movements by a subject according to the Tinetti mobility test requirements which are defined and accepted as following kinesiology standards and protocol: the Tinetti test defined the subjective assessments. The MATT system makes these assessments objective computerized measurements and assessments. The eight selected movements of the subject are: sit still in a chair, arise from sitting in a chair, stand still, stand still with eyes closed, sit down on a chair, walk in a straight path, turn 360 degrees walking in a circle and turn 360 degrees turning on-the-spot. With these eight, simple movements, the administration of the mobility assessment mobility codes of the MATT objectively extract 13 specific features' values measure of mobility parameters with which the mobility assessment determines if the measured numerical values of these features' are within the range of the thresholds set for each feature. Feature values lying outside these thresholds allow additional mobility codes to determine the mobility abnormalities these out-of-range features' and may further determine the possible conditions, illness, injury, pain, disease of the subject indicative of such abnormalities.

In an alternative embodiment images from multiple cameras may be used as shown schematically in FIG. 1 (camera A 103 and camera B 104). One of these cameras could be an infrared illumination source and receiving detector and the other could be a visible detector such as the Microsoft Kinect duel camera system utilized in the Microsoft games console. Both the original Kinect V-1 and the newer version Kinect V-2 have been implemented in this Mobility Assessment Tool (MATT) system (Bunn et al., Gait Assessment Using the Kinect RBB-D Sensor, IEEE Milano Italy, Aug. 25-29, 2015). Each has been found to be an inexpensive 2-camera sensor system with the added advantage of significantly improving separation of the background from the moving image of the subject. The data are composed into a stereoscopic 3-dimensional (3-D) representation of the subject's movements using known image reconstruction techniques, and the Kinect cameras can transform the images of the subject in the video recording to become an isolation of the moving subject with full retention of all movements of all of the subject's body including feet, legs, trunk, arms, hands and head while rendering the recording devoid of the information needed to identify the subject. Additionally, the Kinect video camera system has imbedded software that produces multiple skeleton nodes (20 for the V-1 and 25 for the V-2). Revealed here in we have incorporated into the MATT assessment mobility codes for making the measurements objectively determining the movement of these nodes video frame by video frame to assess the movement of a subject performing the movements of the Tinetti test. The MATT omits the Tinetti nudged a subject sub-assessment as herein it is considered to be an invasive interference of the subject.

For data acquisition, the Kinect sensor samples at a frequency of approximately 30 Hz and video frames are captured both in color and depth. Using captured frames, the middleware of Kinect software SDK, on a frame basis segments the subject's human shape and imposes skeleton nodes on the shape providing in each frame the output of a human skeleton represented by 20 nodes, for the Kinect V-1 and 25 nodes for the V-2, as control points in the Kinect's own reference frame known as the skeleton space. Each node represents a specific joint with 3D position information in units of meters. The skeleton space uses a right-handed coordinate system: the Y axis lies in vertical direction of the image plane, the Z axis extends in depth perpendicularly from the sensor and the X axis is horizontal in the image plane and orthogonal to the Y and Z axes.

In pre-processing, the MATT subjective computerized analysis mobility codes compute the position and the speed of each joint node from frame to frame in the time sequence each of which are considered as one-dimensional signals. The MATT mobility codes apply two 2^{nd} order low-pass Butterworth smoothing filters were used to reduce the noise in the signals. The MATT analysis mobility codes apply empirically-determined cut-off frequencies of 4 Hz and 1 Hz were used for the objectively determined position and speed signals of each joint, respectively.

To extract the features' of walking steps, it is necessary to accurately segment the steps, i.e. determine the start and the end of a step. The Z component (in depth) of foot speed is used because it showed good regularity in relation to the phases of the steps. The MATT mobility codes robustly segments the steps while ignoring the small peaks generated by the interference from parts of the body overlapping or the distance between the subject and the camera being too long.

MATT objective computerized analysis mobility codes determine the time series of the Z speeds of both feet during stepping. The most important features' are the start-, the mid- and the end-points. MATT objective computerized analysis mobility codes determine these as feature points and use them for analyzing the gait. The MATT mobility codes are insensitive to the tilt angle of the Kinect sensor since we they use the Z component (in depth) of foot speed for step segmentation.

MATT analysis mobility codes finding overlaps of the feet in the 360° Turn the analysis uses the same pre-processing step as the gait mobility codes. Since a subject is turning 360° on the spot, it is difficult to segment the steps using the method for the gait analysis. To measure the continuity of a turn, the MATT objective analysis mobility codes identify the skeleton frames in which the speeds of both feet are below a certain speed threshold. Specifically, the speed is defined by the MATT objective analysis mobility codes as the Euclidean norm of X and Z components of the speed of a foot. A group of consecutive skeleton frames below a certain speed threshold indicates that a subject may have paused during a 360° turn. The mobility codes identify pauses during the 360° turn based on a toe-off speed threshold of 0.2 m/s. The time interval of each pause is determined by the MATT objective analysis mobility codes as the difference in timestamp of the first skeleton frame and the last skeleton frame in a group.

Several trunk features' are determined by the MATT objective analysis mobility codes. The stability of the trunk of the body is monitored by two factors: the use of arms for balancing and the lean angle of the trunk in the coronal plane. Additionally, for MATT gait mobility codes, it is necessary for them to calculate the deviation of the base of the spine relative to the traveled path.

It is assumed that at the start of an assessment the subject is not using their arms for balancing and the wrists are placed at the sides of body as directed by the computerized voice instructions. In other words, the wrists are at their resting positions. The distance between wrists is defined by the MATT objective analysis mobility codes as the Euclidean norm of X and Y components of positions of two wrists. The Z component is ignored since the arms typically swing during walking.

During a walk or a 360° turn, when subjects use their arms for balancing or lean the trunk of their body, the distance between the wrists will increase. By MATT objective analysis mobility codes calculating the difference of the distance between the wrists at the resting positions and the distance of wrists during a walk or a 360° turn, the use of arms for balancing can be detected by the MATT objective analysis mobility codes. To illustrate the process, the algorithms mobility codes detect the changes of X distances of two wrists with respect to the origin of the Kinect to measure the interval in which a subject may use an arm for balancing.

The leaning angle of the trunk is defined by the MATT objective mobility codes as the angle between the vector of the trunk (between the center of shoulders and the spine base) and the gravitational vector in the coronal plane. The angle is obtained by the mobility codes calculating the mathematical dot product of these two vectors.

To measure the deviation from the path during a walk, a path vector P is calculated by the MATT objective analysis mobility codes using the position of spine base in the first frame and last frame in a walk. The instantaneous deviation from the path is defined by the MATT objective analysis mobility codes as the perpendicular distance between the position of the base of the spine and the straight line path along vector P.

There are 13 specific features' values extracted by the MATT objective mobility codes from the above measurements that further administration of mobility codes will determine for the Walk Gait assessments. For the gait mobility codes, of interest are the three feature points of each step: the start, the mid and the end. The MATT objective analysis mobility codes feature point contains the timestamp, the position and the speed of the moving foot. The gait specific features' values involved in the gait mobility codes are the following (with units in parentheses):
1. Initiation of gait t1 (in milliseconds):
   time consumed between computerized voice instruction “begin” and start of a walk by a subject;
2. Step Through Length for right foot: 1r (in meters—left);
3. Step Through Length for left foot: 1l (in meters);
4. Mean distance between ankles of two feet when both of them touch the ground during a walk;
5. Step Height for right foot, Sr (in meters per second);
6. for left foot, S1 (in meters per second);
7. Mean Speed of a moving foot in the vertical direction;
8. Step Length for left foot: d1 (in meters): length of left foot step from step-start at heel lift-up to step-stop at heel put-down;
9. Step Length dr (in meters): length of right foot step from step-start at heel lift-up to step-stop at heel put-down;
10. Step stance df (in meters): distance between the left foot heel at heel-down and right foot heel at heel-down;
11. Step Interval t2 (in milliseconds): time consumed between end of a right (or left) foot step and a start of new left (or right) foot step;

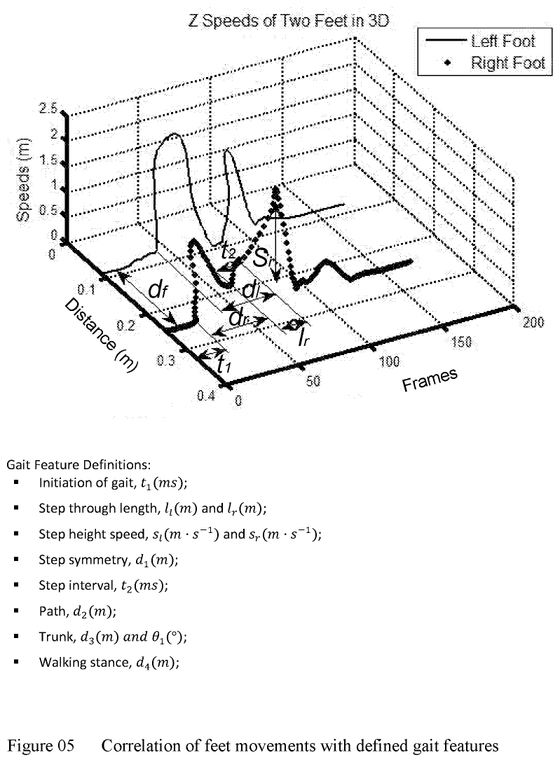

Some of these specific features' values associated with the movements of feet are illustrated in FIG. 5. We define the following features' involved in the 360° turning analysis:
12. Continuity of steps t3 (in milliseconds);
13. Steadiness d5 (in meters)

The classification of normal and abnormal patterns of each gait feature of a subject is performed by setting thresholds for the features' values extracted from the recorded skeleton. To determine the thresholds of the features', data were captured from athletes with potential risk of concussion and a kinesiologist was asked to score the athletes by watching the pre-recorded videos using the software developed for the study. The scores given by the kinesiologist were used as the ground truth for determining the thresholds.

The mobility codes were designed using Matlab 2014a for data analysis and later were redesigned and coded in C++. Using Microsoft Visual Studio 2013, a desktop application was designed for performing experimental real-time assessments and further advancement of the designs has created the MATT as a tool for kinesiology professionals, practitioners and clinical testers to use as the new and validated mobility assessment tool. By way of example, the design methods will now be revealed herein.

In this example data were captured from 14 athlete subjects sample group by researchers in the department of Kinesiology at York University. Three athletes had a history of concussions, one had a suspected concussion and the rest were healthy controls. Informed consent was obtained from the participants in accordance with a protocol approved by the Human Participants Review Subcommittee at York University.

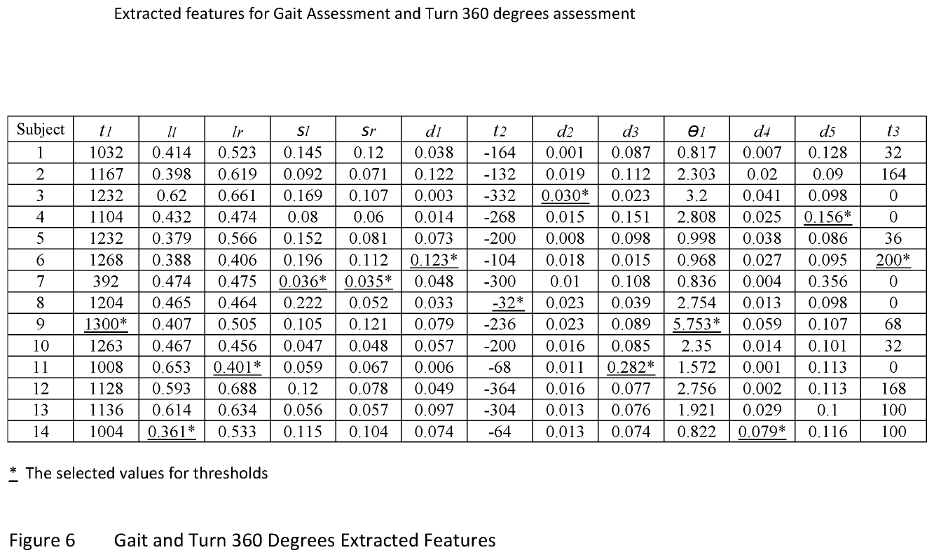

A Kinect V-1 sensor (camera) used was placed 0.84 m above the ground. For gait assessments, the athletes were asked to stand 3.8 m away from the camera, perform a straight line walk towards the camera and stop at 1.8 m away from the camera. For 360° turning assessments, the athletes were asked to stand at a position between 1.8 m and 3.8 m away from the camera and perform a 360° turn. To calibrate the system, the specific features' values to be extracted from the collected data were determined using the developed mobility codes, as shown in the table of FIG. 6. From the table, some interesting patterns can be observed. For example, in the range of features' values from all 14 subjects, it took most subjects more than 1000 mil-seconds (ms) to initiate a walk as shown in the second column t1. Ideally, the sample data should cover all normal and abnormal patterns of gait analysis and 360° turning analysis so that it is possible to determine optimal thresholds for each feature.

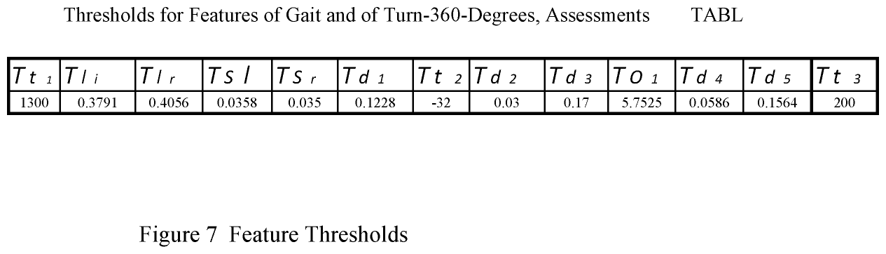

The approach taken to set the thresholds is to consider the 14-subject sample group representative of normal variation. Then, for each feature, the values limit selected is one that will enable all normal participants to pass the automated assessment since all 14 were passed by the kinesiologist's subjective assessment; the limit is normally the value that represents the worst case in a sub-assessment as shown in the table of FIG. 7. For example, in steadiness assessment in 360° turning analysis, the value 0.1564 meter was selected instead of 0.3564 meter because, in the latter case, the subject used arms for balancing which is determined to be abnormal, and which resulted in a longer distance. These features' values thresholds are entered as parameters for the mobility codes in the MATT application in this example, for use in experimental real-time assessments of subjects. These parameter settings form the initial baseline for scoring detection of abnormal gaits determined in follow-up clinical studies and subsequently refined to optimize discrimination of normal and abnormal gait for this particular group.

The primary task for any given subject sample group is to build a database that contains as many samples as possible from relevant clinical populations. When the number of samples is large enough and adequately covers normal and abnormal patterns of each gait feature, the accuracy of the determination and segmentation of normal and abnormal gait is improved and new thresholds and more advanced classification mobility codes can be determined. It will be clear to anyone with a kinesiology understanding that the MATT methods and mobility codes revealed in this patent disclosure, will allow the establishing of databases specialized for clinical populations having particular mobility issues such as related to specific injuries, illnesses, pain, diseases and conditions such as concussion, dementia, chronic pain, Parkinson's, and stroke. The database described by the above example was dealing with male and female subjects in age ranges of 18-25, who are athletic and who have a risk of suffering brain concussions. It will further be clear that due to the objectivity, reliability and reproducibility of the testing mobility of subjects with the MATT system and the mobility codes, that the results from repeated testing with the MATT of subjects will permit the tracking and monitoring over time, of a subject's particular condition and it's progression of improvement or lack of improvement during treatment being given the subject for that condition. The MATT could become as common and fundamental a medical professional tool as the blood pressure measuring tools found in almost every medical practitioner's office to track and monitor patient's heart and blood pressure cardio vascular condition.

In clinical tests of subjects with the MATT mobility assessment system conducted to date to test and validate the assessment methods and apparatus, it was found that the methods and apparatus were well received by the kinesiology professionals as functional and highly accepted as a unbiased, objective and reproducible tool providing valuable patient mobility information. For the linkage relationships determined between current and previous subject's assessments in evaluating the changes in mobility and mobility impairment and potential existence of concussion as well as and for illness, pain or disease curing, arresting or reversing effects of the illness, pain or disease the MATT was also recognized to be effective.

The system described above has the capability to determine relationships of a subject's present assessments to the subject's previous assessments whereby the expert system can determine and measure the changes in any of the actions and motions of the subject specifically tailored to the subject's individual conditions and health. The expert system not only has databases of information on what are considered normal movements and actions of persons depending on age, sex, health condition and drug use, but also has similar databases specific to the subject being assessed, and thus the expert system can also base-line calibrate its decision-making determinations to what are considered normal movements and actions of the subject being assessed. Determining the relationships to the subject's base-line the expert system can further determine if the present assessment is normal or if it indicates a mobility impairment condition and possible potential existence of injury such as concussion, illness, pain or disease. If the system determines that a mobility impairment condition exists, then the system can determine relationships of the present assessment to previous assessments for this subject to further determine changes in the mobility impairment conditions. Further, if video monitoring in areas where the subject moves about, such as in a residence, home, hospital, playing and sports fields, professional stadium and sports entertainment facilities or natural environments are implemented as the earlier discussion noted, the expert system can determine relationships of these data with which the system can determine the mobility impairment and changes in the subject's mobility in the subject's daily living environment from which the system can determine more comprehensive preventative and remedial practices, health and well-being programs, mobility aids, and monitoring programs for improved quality of life activities, work related activities, monitoring of rehabilitation programs and their success or failure or modifications specific for the subject.

In either real-time or post-recording, the MATT expert system can be the decision-making facility which permits the actual operation of the system and assessment to be done by regular staff of the subject's employer, or clinic, or athletic or sports facilities without the need for highly qualified and expensive professional personnel. This frees up the professional practitioners time by integrating the MATT results into the diagnosis of their patient's mobility and health condition. The apparatus and methods described above can also allow authorized personnel, such as professional physiotherapists, neurologists and concussion specialists to review this new source of mobility assessment data and the determinations made by the MATT system, and integrate this information into their diagnosis of their patients' conditions.

A new and unique embodiment of the expert system is revealed here, that for the first time provides a fully computerized automation implementation of the standard kinesiology mobility test fundamentals of the Tinetti test as a tool for the kinesiology professional which provides consistent, reproducible and reliable testing results across any and all testers. Every subject receives identical computer generated verbal and video instructions, each and every time the subject performs the assessment test. This eliminates inter-tester and intra-tester reliability errors. Instructions are in a variety of selectable languages suited to the subject's requirements.

Also, the MATT revealed herein, is designed to save time for both administrators and health care professionals. Assessment results are provided in both gross overall mobility scores and detailed results of the subject's performance of specified movements. Numerical and textual data are provided in readily accessible formats that can quickly and easily be stored and transferred within internal file format frameworks and exported to standardized spreadsheet and word processing formats based on kinesiology practice and setting.

Double blind clinical trails have been conducted at the York University Kinesiology and Health Department, Dr. Lauren Segio and Dr. Diana Gorbet. Testing during the trials was conducted on over 20 of the Canadian Women's and Men's handball team players from the 2015 Canada Summer Games several of whom were considered to likely have suffered injuries and some possible concussions. Also tested were over 70 university athletes considered as normal uninjured subjects. Tests included several standard kinesiology kinematic, cognitive, balance, gait, coordination, and vision tests to determine the physical and mental condition of the subjects.

The objective MATT gait and balance expert system's fully computerized tests described here in were administered to the video taken for each subject as they carry out the Tinetti gain and balance test movements. The expert system analyses of each video produced a Tinetti score for each subject. Also, three independent physiotherapists separately conducted the subjective Tinetti gait balance test scoring for each subject. The physiotherapists were required to make their personal subjective Tinetti test scoring assessments, and were only allowed to view the videos of the subjects movement but not allowed any access to the other double blind test data or results. Only the team of Drs. Sergio and Gorbet has access to all test data and results prior to their publication.

The early results from the clinical trials, indicates that the MATT Tinetti scoring and the independent physiotherapists Tinetti Scoring and the York University Kinesiology testing results are all in good agreement. Further the early results also indicate that the reliability, reproducibility, and consistency of the MATT assessments demonstrated that the variability in the physiotherapy personalized testing strongly supports the need for computerized expert gait and balance technology assessment of athletes, such as the MATT. Final results will be published 2018-19. Publication of results from the comparison of the York University Kinesiology subjective scoring of the SCAT-5 test and the MATT objective scoring of the SCAT-5 test will follow.

From the above it will be clear the assessment methods and apparatus of the MATT tool described could be applied to many environments, such as, hospitals, private homes, hotels, commercial establishments, doctor's offices, clinics, drugstores, mobility-aids stores, and in the broad sense anywhere people are moving about such as sports and athletic facilities, playing fields, gyms, employment facilities. Also it will be clear to anyone versed in the healthcare field that many different mobility codes, mobility codes test parameters, action scoring methods and determinations can be implemented, including, mobility impairment mobility codes, time derivative determinations and mobility testing, such as those we reveal as incorporated into the computer facility active logic engine neural networks decision determinations methods and apparatus with which we can assess mobility impairment and potential existence of injury, illness, pain or disease, the preventative outcomes and recommendations to reduce further mobility impairment and potential further injury, and for improved quality of life for assessed subjects. Further, it will also be clear that the methods and apparatus of the MATT tool, assessments and recommendations facilitated by the expert system can have application to any subject persons regardless of their age, health, sex, location or activity. Also, it will also be clear that the methods and apparatus, assessments and recommendations facilitated by the expert system can have application to assessment of and the tracking the progression of injury such as brain concussion, and the effects of treatments and rehabilitation regimes whether trials or long-term such as drugs, physiotherapy, nutrition, exercise, and success or failure of those treatments, and for other conditions such as diseases, illnesses, pains and injuries not limited to only those disclosed herein.

What we claim is:

1. A computerized system method comprising analysis computer code implemented by at least one first computer having a processor, a memory, data input recording and output capability and coded logic engine implementing a uniquely objective analysis computer code for determining of the mobility and mobility impairment of a subject performing the 8 physical movements of the here-to-fore subjective assessed by a kinesiologist, the kinesiology Tinetti gait and balance risk of fall mobility test, including movements: sit-up straight in a chair; stand up from a chair; stand still; stand still with eyes closed; sit down on a chair; walk in a straight-line path; turn 360 degrees walking in a complete circle; turn 360 degrees turning-on-the-spot, for which method a second computer having a processor, a memory and input/output capability, objectively observes the movements in the visible and the non-visible such as the infrared spectrum from a dual sensor and which second computer outputs these observations as visible and non-visible such as infrared video and as a multi-nodal skeleton video overlay, data streams for which said streams can be at a variety of data rates such as thirty frames per second, which data streams are output to the first computer which has the capability to receive and to record and store in databases the said data streams and has the capability to herein objectively determine and extract and store thirty movement features and known norms of said features from the video and skeleton streams of the movements, including at least thirteen features utilized herein by the said first computer to objectively determine, the here-to-fore subjective, the objective kinesiology Tinetti test.

2. The method of claim 1, wherein the system first computer includes said coded logic engine capable of displaying said video and overlay skeleton data streams from either the live data streams directly from the second computer or playback from the first computer recorded and stored databases of said video and skeleton data, for the observation of the said movements of the said subject, which playback will allow a person to view the data streams and from which the person can subjectively score the said here-to-fore subjective kinesiology Tinetti gait and balance mobility test from viewing said movements from which, following the kinesiology subjective practice and protocols, to produce a subjective movement scoring result such as the Tinetti test gait/balance and risk of falling scoring.

3. The method of claim 1, wherein the system first computer extracted thirty features from which the said thirteen features include: initiation of gait t1 defined in milliseconds; step through length 1r for right foot defined in meters; step through length 1l for left foot defined in meters; step height for right foot Sr defined in meters per second; step height for left foot S1 defined in meters per second; step symmetry d1 defined in meters; step interval t2 defined in milliseconds; path d2 defined in meters; trunk d3 defined in meters; leaning angle θ1 defined in degrees; walking stance d4 defined in meters; continuity of steps t3 defined in milliseconds; steadiness d5 defined in meters.

4. The method of claim 1, wherein said first computer system data analysis code instructs said logic engine to objectively measure and extract said thirty features, frame-to-frame from said data streams, and applies further additional mobility analysis computer code instructions to said logic engine code which code objectively analyzes the said thirteen features and determines the mobility assessment of said subject in accordance with kinesiology practice and protocols by objectively assessing the said kinesiology Tinetti test.

5. The method of claim 1 wherein the system, a first computer determines from administration to said thirteen features by said active logic engine of said uniquely objective video analysis computer code whereby to determine the Tinetti test mobility and mobility impairment of said subject from the application of said objective code to the contents of the said video and skeleton data streams.

6. The method of claim 1, wherein said system comprises said objective analysis computer code, video data handling computer code, vision and machine learning features code instructions applied to said first computer engine for utilizing said sensor's provision of skeleton multi-nodal representation of the said subject's body joints including head, neck, shoulders, elbows, wrists, hands, trunk, hips, knees, ankles; for which administering further objective computerized features code analysis applied to the said skeleton nodal data, the said thirteen features can be extracted from said skeleton nodal data stream by the logic engine implanting the said features code instructions.

7. The method of claim 1, wherein said system comprises said first computer objective analysis computer code, video data handling computer code, vision and learning instructions, and additional uniquely objective skeleton extraction code instructions applied to said second computer engine for said engine to derive from the said senor's visible and non-visible such as infrared video data streams and store in said databases, the said skeleton overlay data stream which administering said further objective computerized features code analysis applied to the said skeleton nodal data, the said thirty movement features can be extracted from said skeleton nodal data stream or from said stored skeleton nodal data streams.

8. The method of claim 1, wherein said system first computer comprises data input recording and output capability and coded logic engine implementing a uniquely objective analysis computer code for determining of the mobility and mobility impairment of a subject performing the 8 physical movements of the here-to-fore subjective kinesiology Tinetti gait and balance mobility test with which said output capability and coded logic said engine can format said results from either subjective kinesiolgist determined or objective computerized determined results from said Tinetti gait and balance mobility test scoring, said outputs can be a variety of formats such as in the standard balance scoring and gait scoring Tinetti format in a variety of output such as hardcopy printout, spreadsheet such as Excel output and computer data file recorded.

9. The method of claim 1, wherein the repetition of said kinesiology Tinetti test over a period of time provides a time sequence of said determinations of said subject's mobility and impairment and said recordings of said data streams and provides recordings of said time sequence of said data streams.

10. The method of claim 9, wherein said recordings of said time sequences, additional computer code and instructions thereby instructs the said first computer to determine, track and record in said databases, over time the said subject's mobility and impairment assessments and the range of said assessments.

11. The method of claim 10, wherein said additional code and instructions to said first computer determines, tracks and records the said subject's mobility and impairment conditions, additional code and instructions instructs the said first computer to track the eventual mobility and impairment outcomes of the said subject.

12. The method of claim 11, wherein said repetition of said kinesiology Tinetti test for each individual subject as members of a group of subjects all of whom are selected as being at a stage where each has similar mobility and impairment assessments, additional computer code and instructions to said first computer determines the collective representation of the mobility and impairments of the said group as a whole for the said stage, and additional code and instructions instructs the said first computer to track the eventual mobility and impairment outcomes of the said subjects and of the group as a whole.

13. The method of claim 12, wherein several groups, each being at different said stages and at different eventual outcomes, additional code and instructions instructs the said first computer to determine the time sequence of each group's stage and outcomes and to compile a database of said stages and outcomes represented by the collective of all said groups, which database forms the standards representative of each stage.

14. The method of claim thirteen, wherein additional code and instructions instructs the said first computer to determine the comparison of the mobility, impairment and outcomes of an individual subject determined at a specific time, with the said database standards of mobility, impairment and outcomes of each stage by which comparison said computer further determines the range in the comparative standard stages at which the said individual subject's mobility and impairments and outcomes, determined at said specific time, matches the said standards.

15. A method according to claim thirteen, wherein for each assessment in said range of assessments additional objective computer code and instructions implemented by said engine, determines a set of stages and conditions representative of said assessments for a range varying from those of poor mobility and high impairment to those of high mobility and low impairment conditions.

16. A method according to claim 15 wherein additional objective computer code and instructions implemented by said engine, determines the comparison of assessments of an individual subject with those assessments of the said range, thereby determining the stage of the subject's gait, balance, mobility and impairment at the time the subject was assessed.

17. A computerized system method comprising analysis computer code implemented by at least one first computer having a processor, a memory, data input recording and output capability and coded logic engine implementing a uniquely objective analysis computer code for determining of the mobility and mobility impairment of a subject performing the physical movements of the here-to-fore kinesiologist subjectively assessed, the kinesiology Sports Concussion Assessment Tool test now in version five as SCAT-5, including some of the movements while standing on a force platform with pressure sensors, for which method a second computer having a processor, a memory and input/output capability, objectively observes the movements in the visible and the non-visible such as the infrared spectrum from a dual sensor, and observes the subject's movements detected by the pressure sensors and which second computer outputs these observations as data streams of visible and non-visible video and as a multi-nodal skeleton video overlay of the subject whether performing on and performing not on the force platform and of pressure sensor data from the subject's feet when performing on the platform, said data streams for which said streams can be at a variety of data rates, and which data streams are output to the first computer which has the capability to receive and to record and store in databases the said data streams and has the capability to objectively determine and extract and store the subject's movement features and known norms of said features from said data streams and with which features the said first computer objectively determines the kinesiology SCAT-5 test mobility and concussion assessments.

18. The method of claim 17, wherein said first computer system data analysis code instructs said logic engine to objectively measure and extract said features from said data streams, and applies further additional mobility analysis computer code instructions to said logic engine code which code objectively analyzes the said features and determines the assessment of said subject in accordance with kinesiology practice and protocols of the said kinesiology SCAT-5 test.

19. The method of claim 17, wherein the system first computer includes said coded logic engine capable of analyzing said video and overlay skeleton and pressure sensor data streams from either the live data streams directly from the second computer or playback from the first computer recorded and stored databases of said video and skeleton and pressure data, for the observation of the said movements of the said subject, which said analyzing by the said coded logic engine implementing a uniquely objective analysis computer code objectively scores the kinesiology SCAT-5 test following the kinesiology SCAT-5 test practice and protocols and produces an objective scoring result such as the SCAT-5 assessment of the possibility of the subject having suffered a brain concussion.

20. The method of claim 17, wherein the system first computer includes said coded logic engine capable of displaying said video and overlay skeleton and pressure sensor data streams from either the live data streams directly from the second computer or playback from the first computer recorded and stored databases of said video and skeleton and pressure data, for the observation of the said movements of the said subject, which playback will allow a person to view the data streams and from which viewing the person can subjectively score the said here-to-fore subjective kinesiology SCAT-5 test from viewing said movements from which, following the kinesiology practice and protocols, the person produces a subjective movement scoring result such as the SCAT-5 possibility of the subject having suffered a brain concussion.
